(19) United States
(12) Patent Application Publication
(10) Pub. No.: US 2019/0204293 A1
(43) Pub. Date: Jul. 4, 2019

(54) SENSOR FOR CHEMICAL ANALYSIS AND METHODS FOR MANUFACTURING THE SAME

(71) Applicant: Life Technologies Corporation, Carlsbad, CA (US)

(72) Inventors: John DONOHUE, Southbury, CT (US); Phil WAGGONER, Guilford, CT (US); Scott PARKER, East Haven, CT (US); Wolfgang HINZ, Killingworth, CT (US); Chiu Tai Andrew WONG, Orange, CT (US)

(21) Appl. No.: 16/229,272

(22) Filed: Dec. 21, 2018

Related U.S. Application Data
(60) Provisional application No. 62/611,453, filed on Dec. 28, 2017.

(51) Int. Cl. G01N 33/487; G01N 27/02; B01L 3/00

(52) U.S. Cl. CPC G01N 33/48721; G01N 27/028; H03K 17/6871; G01N 27/021; B01L 3/5085

(57) ABSTRACT

A chemical sensor for analyte solutions utilizes AC excitation of a sample distributed in one or more micro-wells of a measurement device. The sensors utilize narrowband filtering of the measured signal(s), resulting in a large reduction in noise. Synchronous detection is utilized to provide high discrimination of the desired signal from noise or interfering sources. Conductance and by extension impedance is measured by applying a constant alternating current (AC) voltage across the electrodes of each micro-well and measuring the resulting current.

### CROSS-REFERENCE TO RELATED APPLICATION(S)

This application claims benefit of U.S. Provisional Application No. 62/611,453, filed Dec. 28, 2017, which is incorporated herein by reference in its entirety.

### BACKGROUND

The present disclosure relates to electrical sensors for chemical analysis, and to methods for manufacturing such sensors.

Arrays of chemical sensors may be used to monitor chemical reactions, such as DNA sequencing reactions, based on the detection of ions present, generated, or used during the reactions. More generally, large arrays of chemical sensors may be employed to detect and measure static or dynamic amounts or concentrations of a variety of analytes (e.g. hydrogen ions, other ions, compounds, etc.) in a variety of processes. The processes may for example be biological or chemical reactions, cell or tissue cultures or monitoring neural activity, nucleic acid sequencing, etc.

A change in dielectric or electrical property of the sensor may be measured, for example, by one or more of a change in electrical impedance, capacitance, inductance, conductance or resistance, or a change in resonant frequency. A change in the dielectric or electrical property may be generated from an increase in molecular size or length of a nucleic acid strand present in the area or volume colocated with the sensor. The change may in some cases be an increase due to polymerization (including but not limited to by polymerase addition to DNA or RNA, or by protein synthesis, for example). In other cases the change may be a decrease in the length or molecular size of the nucleic acid or molecule(s) present in the area or volume colocated with the sensor. A decrease may be by attributed to either sequential or non-sequential digestion of the nucleic acid strand (including, but not limited to, exonuclease digestion of DNA or protease digestion of protein). The change may be caused by incorporation of additional molecules or nucleic acids to an existing nucleic acid strand or molecules present in the area or volume in which the sensor is colocated. In some cases the change may be generated from the binding of an antibody to an antigen. The change in some cases may be caused by a disassociation of additional molecules to existing molecules in the area or volume colocated with the sensor. The disassociation may in some cases be the release of a hybridized or bound molecule from another molecule or nucleic acid strand in the area or volume colocated with the sensor.

### BRIEF SUMMARY

### Brief Description of the Several Views of the Drawings

To easily identify the discussion of any particular element or act, the most significant digit or digits in a reference number refer to the figure number in which that element is first introduced.

FIG. 1 illustrates a nucleic acid sequencing system 100 in accordance with one embodiment.

FIG. 2 illustrates a flow cell 200 and micro-well 216 in accordance with one embodiment.

FIG. 3 illustrates an impedance sensor 300 in accordance with one embodiment.

FIG. 4 illustrates a signal processing system 400 for an impedance sensor in accordance with one embodiment.

FIG. 5 illustrates an orthogonal synchronous detector 500 in accordance with one embodiment.

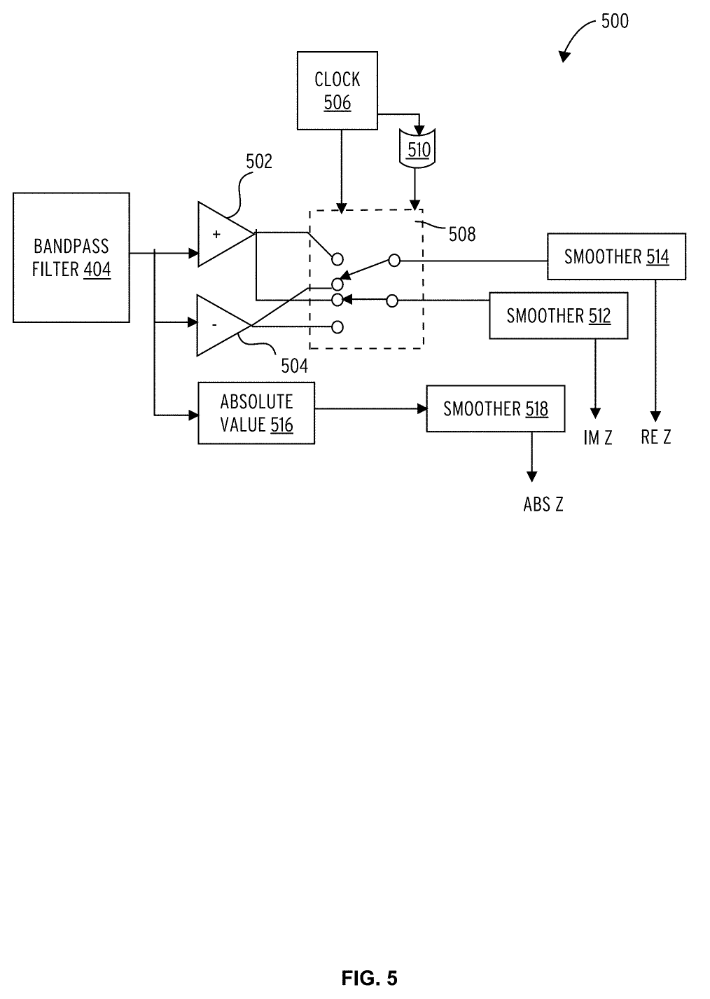

FIG. 6 illustrates a detector real value response 600 in accordance with one embodiment.

FIG. 7 illustrates a detector imaginary response 700 in accordance with one embodiment.

FIG. 8 is an example block diagram of a sensor array controller 108 that may incorporate embodiments of the present invention.

### DETAILED DESCRIPTION

The following detailed description refers to the accompanying drawings that illustrate exemplary embodiments consistent with this invention. Other embodiments are possible, and modifications can be made to the embodiments within the scope of the invention. Therefore, the detailed description is not meant to limit the invention.

It would be apparent to person of ordinary skill in the relevant art that the present invention, as described below, can be implemented in many different embodiments of hardware or the entities illustrated in the figures. Thus, the operational behavior of embodiments of the present invention will be described with the understanding that modifications and variations of the embodiments are possible, given the level of detail presented herein.

In some implementations, the impedance of a sample may be measured using a periodic signal generated across the sensor electrodes and through the sample to measure. The period signal may comprise a single frequency, or multiple frequencies. In some cases, the excitation signal between the electrodes is a complex waveform. Two or more frequencies or excitation patterns may be added or applied concurrently to the sample. Alternatively, two or more frequencies or excitation patterns can be applied consecutively, or excitation patterns may include portions that are concurrent and consecutive. By way of example, the frequencies may be selected from a range of 10 Hz to 1 MHz, 70 Hz to 1 MHz, 100 Hz to 500 kHz, or 100 Hz to 10 kHz. The excitation pattern may include a sinusoidal pattern, square pattern, saw tooth pattern, or any of various other periodic forms, or a combination thereof.

Arrays of chemical sensors may be colocated in micro-wells of a flow chamber where analyte reactions take place, to detect or identify characteristics or properties of an analyte of interest. An analyte (e.g. DNA) is loaded between electrodes, whereby a modulation of conductance through the analyte can be measured. The analyte may serve as the basis for or contribute to the charge in the solution between the plates (counter ions being the charge carriers). For example, an analyte physically present between the two electrodes may serve as the basis for or contribute to the signal.

The analyte may be manufactured in the micro-well either with or without a solid support through any suitable manufacturing method. The volume, shape, aspect ratio (such as base width-to-well depth ratio), and other dimensional characteristics of the micro-wells may be selected based on the nature of the reaction taking place, as well as the reagents, byproducts, or labeling techniques (if any) that are employed. Preparing the analyte sample may include depositing copies of a biomolecule analyte into the micro-well. In many cases, solid supports (such as hydrogel particles) including a monoclonal population of analyte molecules may be deposited into the micro-well. The micro-well may include a conformal hydrogel network onto which a monoclonal population of the analyte molecule is generated. An analyte molecule may in some cases be and attached to surface agents within the micro-well. The analyte molecule may be a nucleic acid which is amplified using polymerase chain reaction (PCR), recombinase polymerase amplification (RPA), rolling circle amplification, other amplification techniques, or any combination thereof. Additionally, a primer and an enzyme or polymerase may be applied to the nucleic acid to facilitate nucleotide or probe incorporation or chain extension.

An electrical characteristic of the analyte, for example impedance, may be detected by a chemical sensor colocated in the micro-well(s) with the analyte. The impedance may be measured in a system that lacks a redox reaction, or the system may be designed to incorporate a redox reaction.

An analyte may be supported by a solid support. In some cases, only a single copy of an analyte may be present, or alternatively, multiple copies of an analyte may be attached to a solid phase support. Only one type of analyte may be attached to the solid support (monoclonal) or multiple sample types may be attached to the solid support (polyclonal). By way of example, the solid phase support may be a particle, microparticle, nanoparticle, a bead or a gel. The solid support may be porous or non-porous. Any form of solid support suitable to the reaction may be used.

An issue that arises in the operation of large-scale sensor arrays is the susceptibility of the sensor output signals to noise. For example, the noise affects the accuracy of the downstream signal processing used to determine the characteristics of the chemical or biological process being detected by the sensors. Also, byproducts of the chemical or biological process being detected are produced in small amounts or rapidly decay or react with other constituents.

The sensor embodiments disclosed herein may be used to analyze the nature of biomolecules, such as nucleic acids or proteins. For example, copies of a molecule may be deposited into a micro-well, and changes in the dielectric or electrical characteristics in response to specific changes in the molecule may be used to determine characteristics of the molecules. The dielectric or electrical characteristic detected may include a change in the impedance, capacitance, inductance, conductance or resistance, or a change in resonant frequency.

In some embodiments, chemical sensors for analyte solutions may utilize AC excitation of a sample distributed in one or more micro-wells of a measurement device. The sensors may benefit from narrowband filtering of the measured signal(s), resulting in a large reduction in noise. Synchronous detection is utilized to provide high discrimination of the desired signal from noise or interfering sources. In some embodiments, conductance and by extension impedance is measured by applying a constant alternating current (AC) voltage across the electrodes of a micro-well and measuring the resulting current. Obtaining accurate, high-value resistance may be difficult in integrated circuits. Accordingly, in some embodiments, a current/voltage converter circuit may be provided.

Current excitation may be preferred for an integrated circuit implementation. Current sources may be more easily implemented in semiconductor technology, and large numbers of identical current sources (e.g., for many micro-wells) may be provided using only one transistor per source. The voltage and by extension the impedance appearing across a current source may be measured directly or amplified. In some embodiments, a double-layer interface between a solid support bead and an electrolyte fluid can have a complex impedance, such as, for example, capacitance in addition to conductance. Sensor plate interfaces in each micro-well may have capacitive effects. Thus, the use of AC excitation may provide another dimension of measurement, by measuring at different frequencies, e.g. electrochemical impedance spectroscopy (EIS). This may be performed on a semiconductor chip, using synchronous rectification, multiplying the measured signal with two orthogonal phases of the source frequency, averaging the results, and thereby getting two values (real and imaginary components of the impedance) at each measured frequency and well. This can provide measurement of the complex frequency response while providing high noise rejection. Assuming the low pass filter averages 100s of cycles of the AC signal, noise reduction may exceed 20 dB.

Embodiments of the sensors may utilize full-wave detection of the measured signals or combining synchronous detection or full-wave detection with pre-filters. The impedance of a concentration of charge carriers around a solid support (herein referred to as the resistance Rsens) is determined to a first order. The solid support may preferably be a bead. The resistance Rsens will decrease as the charge increases with increasing extension of DNA molecules concentrated around the solid support. Temporal variation in the value of Rsens due to charge mobility around or throughout the solid support may be modeled as Gaussian variation. An AC current is applied to Rsens, generating a multiplicative noise component to the measured value, e.g., a multiplier of the measured AC voltage across Rsens. Additional additive noise is introduced by the sensor surface and measurement transistors, which has a 1/f characteristic (meaning the SNR decreases as the frequency of the AC excitation decreases).

Effects on detected output are different for multiplicative noise (variation in measured impedance) and additive noise. For equivalent integration times, using a synchronous detector yields better performance than using a relaxation oscillator and counter, due primarily to counter resolution. The synchronous detector yields better performance than a full-wave rectifier and is often easier to integrate into an IC (integrated circuit). Utilizing a band-pass or high-pass filter prior to applying a full-wave rectifier can improve performance, yielding results similar to a synchronous detector. However, the synchronous detector allows detection of real and imaginary components of impedance, providing more information. An overall SNR of ˜10 may yield accurate detection of a 1% variation in impedance. The required SNR and detectable resolution are typically proportional.

The type of the signal noise (1/f or Gaussian) and the source of the noise (additive from the sensor components or multiplicative from temporal Rsens variations) each impact the sensor's performance. Post-detection averaging typically provides improved performance over the use of pre-detection filters.

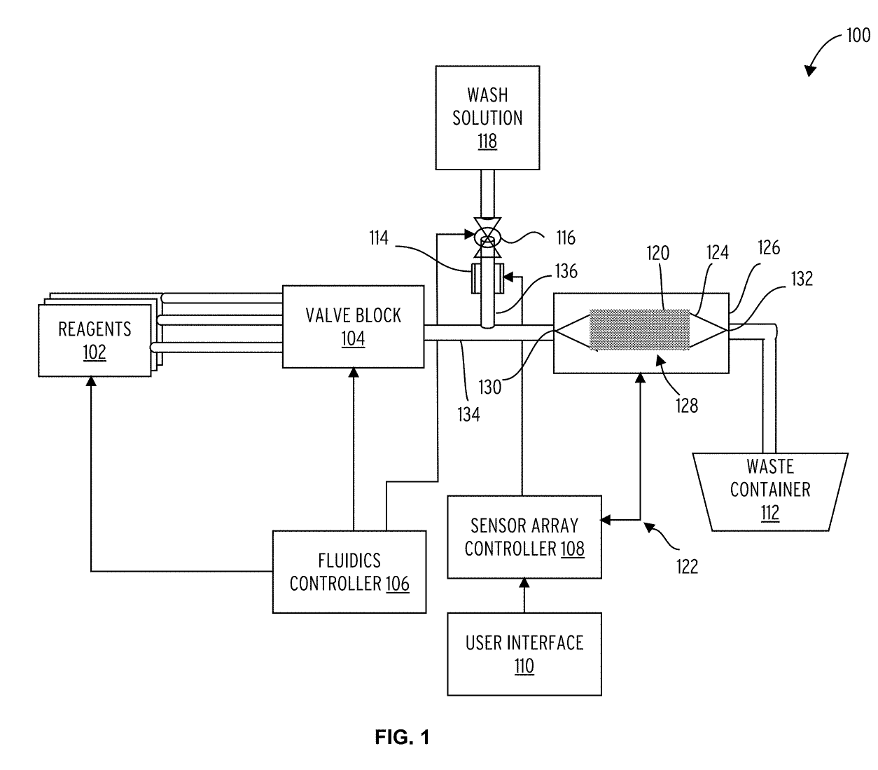

Referring to FIG. 1, a nucleic acid sequencing system 100 includes reagents 102, a valve block 104, a fluidics controller 106, a sensor array controller 108, a user interface 110, a waste container 112, a bias electrode 114, a valve 116, a wash solution 118, and an integrated circuit device 126. The integrated circuit device 126 includes a flow cell 128 comprising a micro-well array 120, a flow chamber 124, an inlet 130, and an outlet 132.

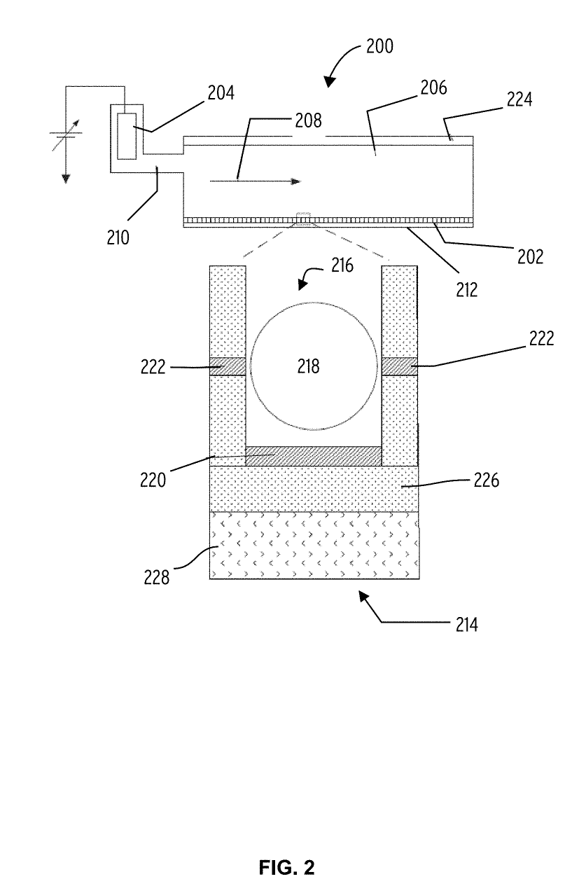

The micro-well array 120 overlays a sensor array (see FIG. 2) that includes sensors as described herein. The inlet 130, flow chamber 124, and the outlet 132 define a flow path for reagents 102 through the flow cell 128 and over and into the micro-well array 120.

The bias electrode 114 may be of any suitable type or shape, including a concentric cylinder with a fluid passage or a wire inserted into a lumen of the passage between the wash solution 118 and the flow cell 128. The reagents 102 may be driven through the fluid pathways of and between the valve block 104 and flow cell 128 by pumps, gas pressure, or other suitable methods, and can be discarded into the waste container 112 after exiting the outlet 132 of the flow cell 128. The fluidics controller 106 controls driving forces for the reagents 102 and also operates the valve 116 and valve block 104.

The micro-well array 120 includes multiple micro-well reaction regions each operationally associated with corresponding sensors in the sensor array (see FIG. 2). For example, each reaction region may be coupled to or incorporate a sensor suitable for detecting an analyte or reaction property of interest within that reaction region. The micro-well array 120 may be integrated within the integrated circuit device 126, so that the flow cell 128 and the associated sensors are packaged into a single device or chip. The flow cell 128 may have a variety of configurations for controlling the path and flow rate of reagents 102 over the micro-well array 120. The sensor array controller 108 provides bias voltages, timing, and control signals to the integrated circuit device 126 for reading the sensors of the sensor array. The sensor array controller 108 also provides a reference bias voltage to the reference electrode 114 to bias the reagents 102 flowing to the micro-well array 120.

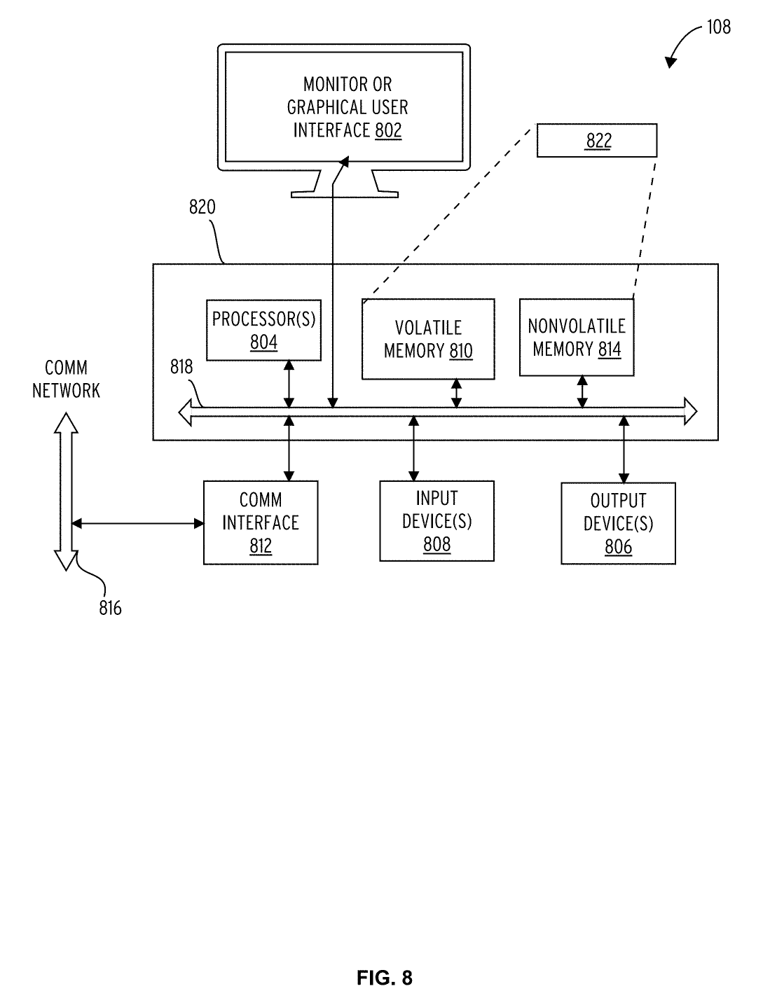

During an experiment, the sensor array controller 108 collects and processes output signals from the sensors of the sensor array through output ports on the micro-well array 120, via a bus 122. The sensor array controller 108 may be a computing device, of various types. The sensor array controller 108 may include memory for storage of data and software applications, a processor for accessing data and executing applications, and components that facilitate communication with the various components of the system (e.g., see FIG. 8).

The values of the output signals of the sensors indicate physical or chemical parameters of one or more reactions taking place in the corresponding reaction regions in the micro-well array 120. The user interface 110 may display information about the flow cell 128 and the output signals received from sensors in the sensor array on the integrated circuit device 126. The user interface 110 may also display instrument settings and controls, and allow a user to enter or set instrument settings and controls.

In some embodiments, during the experiment, the fluidics controller 106 may control delivery of the individual reagents 102 to the flow cell 128 and integrated circuit device 126 in a predetermined sequence, for predetermined durations, at predetermined flow rates. The sensor array controller 108 may then collect and analyze the output signals of the sensors indicating chemical reactions occurring in response to the delivery of the reagents 102. During operation, the system may also monitor and control the temperature of the integrated circuit device 126, so that reactions take place and measurements are made at a known predetermined temperature. The system may be configured to let a single fluid or reagent contact the bias electrode 114 throughout an entire multi-step reaction during operation.

The valve 116 may be shut to prevent any wash solution 118 from flowing into passage 134 as the reagents 102 are flowing. Although the flow of reagents 102 can be stopped, there may be uninterrupted fluid and electrical communication between the bias electrode 114, passage 134, and the micro-well array 120. The distance between the bias electrode 114 and the junction between passage 134 and passage 136 may be selected so that little or no amount of the reagents 102 flowing in passage 134 and possibly diffusing into passage 136 reach the bias electrode 114. In some embodiments, the wash solution 118 may be selected as being in continuous contact with the bias electrode 114, which can be especially useful for multi-step reactions using frequent wash steps.

Referring to FIG. 2, a flow cell 200 comprises a flow chamber 206 having a flow cell cover 224, a reagent flow 208 from a passage 210 in contact with a bias electrode 204, and a sensor array 212 underlying a micro-well array 202. In a breakout view, a micro-well 216 of the micro-well array 202 includes a sensor 214 comprising an electrode 220, a reference electrode 222, a dielectric 226, and a substrate 228. A solid support 218 for an analyte is illustrated inside the micro-well 216 (not necessarily to scale). The bias electrode 204 may not be present in some embodiments.

For solid support in some measurements, a bead having an attached nucleic acid sequence may be utilized. The nucleic acid sequence may be DNA, e.g. single stranded DNA. The bead having the nucleic acid sequence may be, for example, a porous hydrogel, or a solid particle with a hydrogel or similar coating, or a solid particle with DNA directly attached to the surface. DNA may also be immobilized on a hydrogel or polymer coating located between the electrodes or on the surface of one or both of the electrodes. The number of copies of nucleic acid sequences on the solid support bead may be increased by any suitable amplification method including, but not limited to, rolling circle amplification (RCA), exponential RCA, RPA, emPCR, qPCR, or like techniques.

The nucleic acid strands that attach to the bead have an inherent charge. As a nucleotide is incorporated into the nucleic acid strands, the presence of the nucleic acid changes the charge associated with the bead via the nucleic acids. As the bead's charge increases, when immersed in a solution, the available charge in a Debye length around the bead increases, and the conductivity in this region can grow proportionally with the bead's charge, and therefore proportional to the length of the DNA extension.

In some embodiments, the sensor 214 may take measurements, and then a change may be generated in the sample to measure, and additional measurements taken. The two sets of measurements may then be compared to yield information about the composition or characteristics of the sample.

In some embodiments, the molecular size of the biomolecule or a charge of the biomolecule may be manipulated. Specific probes may be added to the biomolecule or the biomolecule may be cleaved. Where the biomolecules includes a nucleic acid or protein, the molecular size may be increased by polymerization, for example, by nucleotide addition to DNA or RNA or protein synthesis. In a particular example, the size of a biomolecule may be increased, for example, by extension of a primer and incorporation of a nucleotide or using a ligation probe. In particular, one of a set of nucleotides may be applied through flow cell 128 of the system and incorporated along the nucleic acid depending on the sequence of the nucleic acid. Optionally, the nucleic acid probe, nucleotide or primer may utilize the ribose or deoxyribose nucleotides, protein analogs or other analogs thereof, or a combination thereof.

In some cases the molecular size of the biomolecules may be decreased. For example, the molecular size may be decreased by sequential or non-sequential digestion, for example, by exonuclease digestion of a nucleic acid or by protease digestion of protein.

The molecular size may also be altered by the association of additional molecules, such as binding probes or moieties, to the biomolecules. For example, the molecular size may be manipulated by applying a moiety to an existing molecule, for example, by hybridization of an oligonucleotide to DNA or RNA or of an antibody or antigen to the biomolecule.

The dissociation of additional molecules may be used to alter the molecular size of the biomolecules, for example, the dissociation or release of hybridize or bound probes.

The sample may be tested to determine a change in the electrical characteristic in response to the change made to the sample. The electrical characteristic may be detected, such as detecting impedance using frequencies as have been described previously.

In some embodiments, the detection of the electrical characteristic may take place in low ionic strength solutions. For example, the ionic strength of the solution may be equivalent to a saline solution having a concentration of 10 μM to 1 mM, such as 10 μM to 100 μm, 10 μM to 90 μM or 10 μM to 70 μM.

The characteristic of the samples, such as a characteristic of biomolecules, may be detected based on the change in the electrical characteristic. For example, a change in impedance in response to the incorporation of a nucleotide may be used to detect the sequence of a nucleic acid. In another example, the association or dissociation of an oligonucleotide probe to a nucleic acid sample may be detected based on a change in impedance and may indicate the presence or absence of a specific sequence within the nucleic acid sample.

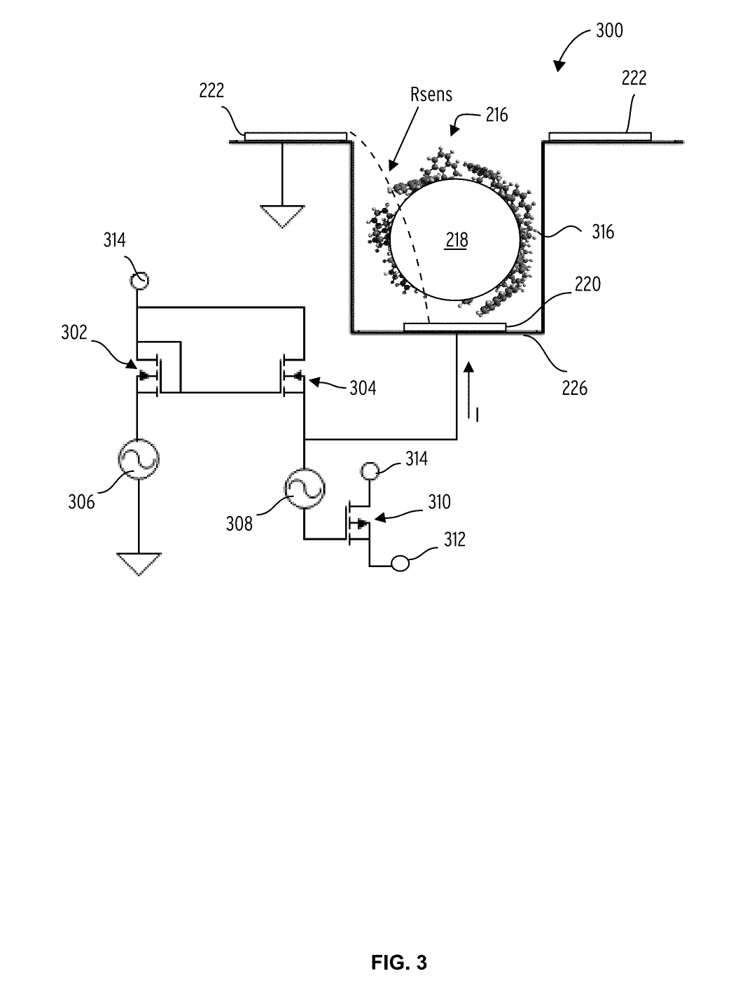

Referring to FIG. 3, an impedance sensor 300 is disposed in a micro-well 216 having a solid support 218 therein, the solid support 218 having attached analyte molecules 316. While the analyte molecules are illustrated as residing on the surface of the solid support 218, analyte molecules, such as RNA or DNA, can be disposed throughout the solid support 2018. For example, the solid support may be porous or may be a hydrogel. The micro-well 216 includes an electrode 220, a reference electrode 222, and a dielectric 226 as previously described in conjunction with FIG. 2.

The impedance sensor 300 utilizes an excitation circuit that includes a P-channel MOSFET 302, a P-channel MOSFET 304, and an N-channel MOSFET 310 with an output terminal 312. The AC excitation circuit is powered from supply voltage terminals 314. An AC excitation signal 306 is generated, producing a drive current I to the electrode 220, which in turn produces a voltage differential between the electrode 220 and the reference electrode 222. The impedance to measure, Rsens, is the ratio of the drive current I and the voltage differential between the electrodes. The output of N-channel MOSFET 310 is proportional to the voltage produced across the impedance Rsens by the current I and is thus proportional to Rsens. During operation, an inherent 1/f noise signal 308 is produced by the various electrical components. In addition, a multiplicative noise component is generated by motion of the analyte molecules 316 or the solid support 218.

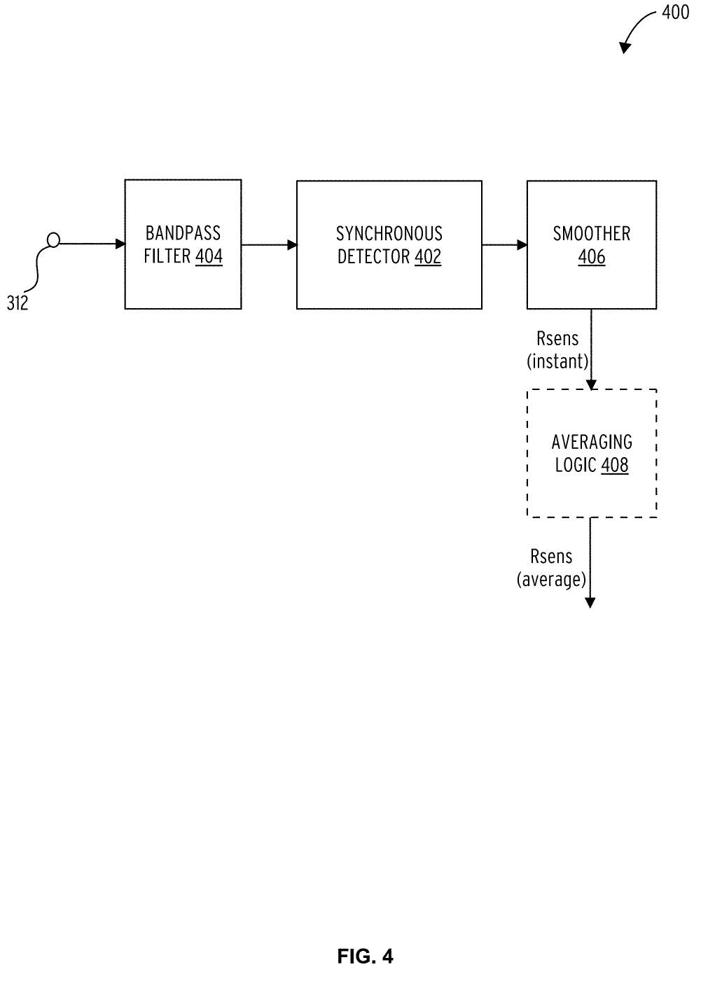

Referring to FIG. 4, a signal processing system 400 for an impedance sensor (e.g., impedance sensor 300) receives a signal from the output terminal 312 of the impedance sensor and applies the signal to a bandpass filter 404. The output of the bandpass filter 404 is applied to a synchronous detector 402, and the output of the synchronous detector 402 is applied to a smoother 406. The bandpass filter 404 may not be present in some embodiments.

The synchronous detector 402 circuit is typically driven by a clock which is synchronous with the AC excitation, and which opens the transistors at the correct times to allow current to flow in the correct direction. The gates are switched on at precise times to allow current in one direction, and precisely switched off to block current from flowing the opposite direction. The output of the synchronous detector is applied to smoothing circuits, typically comprising smoothing capacitors.

Circuitry or signal processing logic (e.g., Digital Signal Processor software or firmware) to implement the bandpass filter 404 will be readily apparent to one of ordinary skill in the art, and will vary with the implementation (e.g., with the desired excitation frequency (f) of the AC excitation signal 306). In embodiments that use the bandpass filter 404, the lower cutoff frequency may be set below f, and the upper cutoff frequency may be set above f. In one embodiment, the cutoff frequencies of the bandpass filter 404 are set to ½ f and 2f. For example, the AC excitation signal 306 may be 10 kHz, and the filter have a lower cutoff frequency of 5 kHz, and an upper cutoff frequency of 20 kHz.

The smoother 406 may be implemented as a resistor-capacitor integrator (series resistance, shunt capacitance). The choice of bandpass filter 404 bandwidth or the smoother 406 time constant may vary with the chosen sensor frequency and the desired measurement speed. Narrower filters and longer time constants will provide better noise reduction and accuracy, at the expense of slower measurement speed.

The synchronous detector 402 implements orthogonal synchronous detection of the sensed impedance. A preferred embodiment utilizes CMOS circuitry in an integrated chip suitable to support a large scale micro-well array 202 each including a sensor 214. SNR characteristics of the synchronous detector 402 are dependent on details of the micro-well 216 dimensions, current drive levels, and sensor 214. The synchronous detector 402 may be designed in manners well known in the art, for example using an amplifier 502, an inverting amplifier 504, and switches 508 driven from a clock 506 directly and via a phase shifter 510. In an example, the detector 402 includes two switches; one with an output representative of a real component of the impedance signal and another with an output representative of an imaginary output of the impedance signal. The clock 506 or phase shifter 510 cause the switches to toggle between an output of the amplifier and the output of the inverter.

Outputs of the amplifier 502 and inverting amplifier 504 may be input to different smoothing logic, one (smoother 514) for the real component of the impedance, one (smoother 512) for the imaginary component of the impedance, and one (smoother 518) providing the absolute value of the impedance. The absolute value 516 logic may be implemented for example as a full-wave rectifier.

In addition to the smoother 406, the signal processing system 400 may utilize post-detection averaging logic (i.e., logic to compute an average of the detected Rsens values).

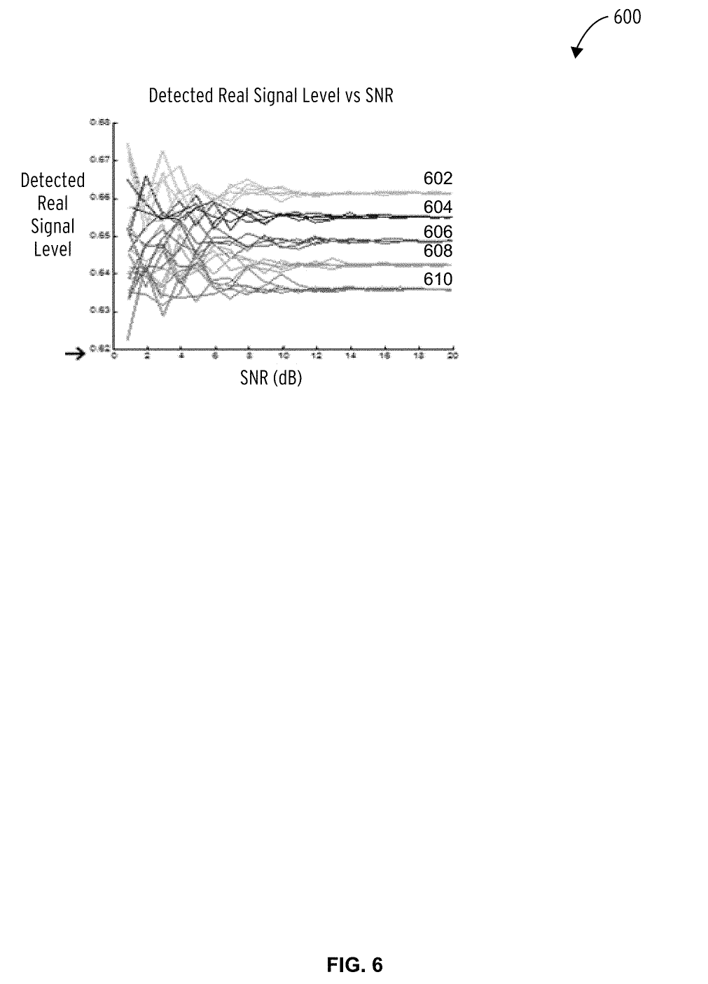

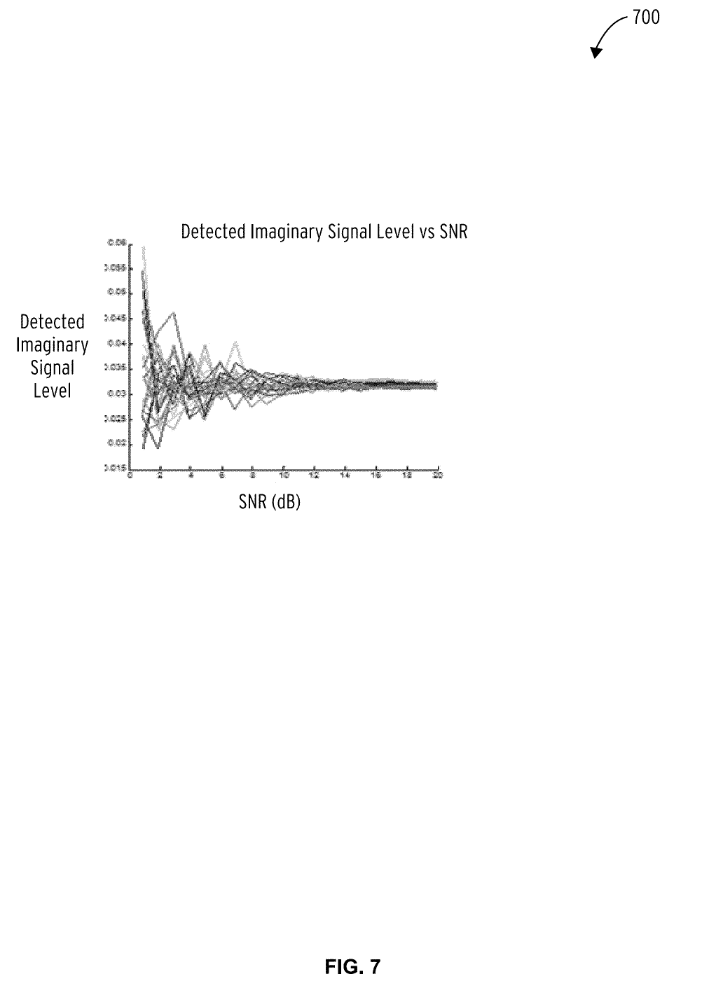

Referring to FIG. 6, a detector real value response 600 is illustrated as a detected signal level vs SNR plot. The different colors (items 602, 604, 606, 608, and 610) represent differing input signal levels each combined with differing amounts of noise (yielding variation in SNR). The detector real value response 600 illustrates the SNR necessary to reliably distinguish different signal levels (e.g., SNR of approximately 10). FIG. 7 illustrates a detector imaginary response 700 for the same detector.

FIG. 8 is an example block diagram of a sensor array controller 108 that may incorporate embodiments of the present invention. FIG. 8 is merely illustrative of a machine system to carry out aspects of the technical processes described herein and does not limit the scope of the claims. One of ordinary skill in the art would recognize other variations, modifications, and alternatives. The sensor array controller can take the form of dedicated computational circuitry, a computer, a tablet, or other computational platform. In one embodiment, the sensor array controller 108 typically includes a monitor or graphical user interface 802, a data processing system 820, a communication network interface 812, input device(s) 808, output device(s) 806, and the like.

As depicted in FIG. 8, the data processing system 820 may include one or more processor(s) 804 that communicate with a number of peripheral devices via a bus subsystem 818. These peripheral devices may include input device(s) 808, output device(s) 806, communication network interface 812, and a storage subsystem, such as a volatile memory 810 and a nonvolatile memory 814.

The volatile memory 810 or the nonvolatile memory 814 may store computer-executable instructions and thus forming logic 822 that when applied to and executed by the processor(s) 804 implement embodiments of the processes disclosed herein.

The input device(s) 808 include devices and mechanisms for inputting information to the data processing system 820. These may include a keyboard, a keypad, a touch screen incorporated into the monitor or graphical user interface 802, audio input devices such as voice recognition systems, microphones, and other types of input devices. In various embodiments, the input device(s) 808 may be embodied as a computer mouse, a trackball, a track pad, a joystick, wireless remote, drawing tablet, voice command system, eye tracking system, and the like. The input device(s) 808 typically allow a user to select objects, icons, control areas, text and the like that appear on the monitor or graphical user interface 802 via a command such as a click of a button or the like.

The output device(s) 806 include devices and mechanisms for outputting information from the data processing system 820. These may include speakers, printers, infrared LEDs, and so on as well understood in the art.

The communication network interface 812 provides an interface to communication networks (e.g., communication network 816) and devices external to the data processing system 820. The communication network interface 812 may serve as an interface for receiving data from and transmitting data to other systems. Embodiments of the communication network interface 812 may include an Ethernet interface, a modem (telephone, satellite, cable, ISDN), (asynchronous) digital subscriber line (DSL), FireWire, USB, a wireless communication interface such as BlueTooth or WiFi, a near field communication wireless interface, a cellular interface, and the like.

The communication network interface 812 may be coupled to the communication network 816 via an antenna, a cable, or the like. In some embodiments, the communication network interface 812 may be physically integrated on a circuit board of the data processing system 820, or in some cases may be implemented in software or firmware, such as “soft modems”, or the like.

The sensor array controller 108 may include logic that enables communications over a network using protocols such as HTTP, TCP/IP, RTP/RTSP, IPX, UDP and the like.

The volatile memory 810 and the nonvolatile memory 814 are examples of tangible media configured to store computer readable data and instructions to implement various embodiments of the processes described herein. Other types of tangible media include removable memory (e.g., pluggable USB memory devices, mobile device SIM cards), optical storage media such as CD-ROMS, DVDs, semiconductor memories such as flash memories, non-transitory read-only-memories (ROMS), battery-backed volatile memories, networked storage devices, and the like. The volatile memory 810 and the nonvolatile memory 814 may be configured to store the basic programming and data constructs that provide the functionality of the disclosed processes and other embodiments thereof that fall within the scope of the present invention.

Software that implements embodiments of the present invention may be stored in the volatile memory 810 or the nonvolatile memory 814. Said software may be read from the volatile memory 810 or nonvolatile memory 814 and executed by the processor(s) 804. The volatile memory 810 and the nonvolatile memory 814 may also provide a repository for storing data used by the software.

The volatile memory 810 and the nonvolatile memory 814 may include a number of memories including a main random access memory (RAM) for storage of instructions and data during program execution and a read only memory (ROM) in which read-only non-transitory instructions are stored. The volatile memory 810 and the nonvolatile memory 814 may include a file storage subsystem providing persistent (non-volatile) storage for program and data files. The volatile memory 810 and the nonvolatile memory 814 may include removable storage systems, such as removable flash memory.

The bus subsystem 818 provides a mechanism for enabling the various components and subsystems of data processing system 820 communicate with each other as intended. Although the communication network interface 812 is depicted schematically as a single bus, some embodiments of the bus subsystem 818 may utilize multiple distinct busses.

It will be readily apparent to one of ordinary skill in the art that the sensor array controller 108 may couple to the sensors described herein via an input device 808 or output device 806 and may be implemented for example by a mobile device such as a smartphone, a desktop computer, a laptop computer, a rack-mounted computer system, a computer server, or a tablet computer device. As commonly known in the art, the sensor array controller 108 may be implemented as a collection of multiple networked computing devices. Further, the sensor array controller 108 will typically include operating system logic (not illustrated) the types and nature of which are well known in the art.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

What is claimed is:

1. A sensing device comprising:
a fluid chamber comprising a plurality of wells;
each well associated with a first electrode and a second electrode, the electrodes positioned to provide an AC excitation through the well;
a synchronous detector electrically coupled to the first electrode and the second electrode; and
the synchronous detector adapted to transform a circuit response to the AC excitation into representations of a real component and an imaginary component of an impedance signal received from the first electrode and the second electrode.

2. The sensing device of claim 1, further comprising a driver circuit to provide the AC excitation to the plurality of wells.

3. The sensing device of claim 2, wherein the driver circuit comprises an input stage comprising a current generator generating the AC excitation as an AC current to the first electrode.

4. The sensing device of claim 2, wherein the driver circuit comprises an output stage coupled to the first electrode, the output stage providing an output representing a sensed voltage generated by the AC current.

5. The sensing device of claim 2, further comprising:
a bandpass filter configured between the output stage and the synchronous detector, the bandpass filter having a center frequency equal to a frequency of the AC excitation.

6. The sensing device of claim 5, wherein the bandpass filter has a lower bound of ½ the frequency of the AC excitation.

7. The sensing device of claim 5, wherein the bandpass filter has an upper bound of twice the frequency of the AC excitation.

8. The sensing device of claim 1, further comprising a smoother connected to an output of the synchronous detector.

9. The sensing device of claim 1, wherein the smoother comprises:
first smoothing logic configured to receive the real component of the impedance output by the synchronous detector; and
second smoothing logic configured to receive the imaginary component of the impedance output by the synchronous detector.

10. The sensing device of claim 1, where the excitation comprises a combination of multiple AC excitation frequencies, and synchronous detectors adapted to detect each of the multiple frequencies

11. The sensing device of claim 1, where the detected circuit responses are generated by biological changes in the contents of the micro-well.

12. The sensing device of claim 11, where the sensed biological change comprises changes in extension of single-stranded DNA to double-stranded DNA.

13. The sensing device of claim 1, wherein the synchronous detector is adapted to transform the circuit response to the AC excitation into an absolute value of the impedance signal.

14. The sensing device of claim 13, wherein the absolute value of the impedance signal is determined using a full-wave rectifier.

15. The sensing device of claim 14, further comprising a smoother coupled to an output of the full-wave rectifier.

16. The sensing device of claim 1, wherein the synchronous detector includes an amplifier, and inverter, and switches.

17. The sensing device of claim 16, wherein the synchronous detector further includes a clock to control the switches.

18. The sensing device of claim 17, wherein the clock is to control the switches directly and through a phase shifter.

19. The sensing device of claim 17, wherein the clock is synchronized with the AC excitation signal.

20. The sensing device of claim 17, wherein a first switch is to switch between connecting the amplifier to a first smoother and connecting the inverter to the firsts smoother to generate the real component of the impedance signal.
